(19) United States
(12) Patent Application Publication
(10) Pub. No.: US 2022/0165398 A1
(43) Pub. Date: May 26, 2022

(54) TREATMENT PLAN IDENTIFICATION

(71) Applicant: Micron Technology, Inc., Boise, ID (US)

(72) Inventors: Rosa M. Avila-Hernandez, Boise, ID (US); Fatma Arzum Simsek-Ege, Boise, ID (US); Kathryn H. Russo, Boise, ID (US); Yixin Yan, Boise, ID (US)

(21) Appl. No.: 17/102,818

(22) Filed: Nov. 24, 2020

(51) Int. Cl. G16H 40/20; G16H 40/67; G16H 10/60; G16H 50/20; G16H 70/20; A61B 5/00

(52) U.S. Cl. CPC G16H 40/20; G16H 40/67; G16H 10/60; G16H 20/10; G16H 70/20; A61B 5/0022; G16H 50/20

(57) ABSTRACT

Methods, apparatuses, and non-transitory machine-readable media associated with treatment plan identification are described. Treatment plan identification can include receiving first signaling from a radio in communication with a second processing resource configured to monitor health data of a patient, receiving at the first processing resource second signaling from a radio in communication with a second processing resource configured to monitor data associated with a plurality of health care providers, writing from the first processing resource to a memory resource data that is based at least in part on a combination of the first and the second signaling, identifying output data representative of a health treatment plan for the patient based at least in part on input data representative of written information and generic health information, and transmitting the output data representative of the health treatment plan.

### TECHNICAL FIELD

The present disclosure relates generally to apparatuses, non-transitory machine-readable media, and methods associated with treatment plan identification.

### BACKGROUND

Memory resources are typically provided as internal, semiconductor, integrated circuits in computers or other electronic systems. There are many different types of memory, including volatile and non-volatile memory. Volatile memory can require power to maintain its data (e.g., host data, error data, etc.). Volatile memory can include random access memory (RAM), dynamic random-access memory (DRAM), static random-access memory (SRAM), synchronous dynamic random-access memory (SDRAM), and thyristor random access memory (TRAM), among other types. Non-volatile memory can provide persistent data by retaining stored data when not powered. Non-volatile memory can include NAND flash memory, NOR flash memory, and resistance variable memory, such as phase change random access memory (PCRAM) and resistive random-access memory (RRAM), ferroelectric random-access memory (FeRAM), and magnetoresistive random access memory (MRAM), such as spin torque transfer random access memory (STT RAM), among other types.

Electronic systems often include a number of processing resources (e.g., one or more processing resources), which may retrieve instructions from a suitable location and execute the instructions and/or store results of the executed instructions to a suitable location (e.g., the memory resources). A processing resource can include a number of functional units such as arithmetic logic unit (ALU) circuitry, floating point unit (FPU) circuitry, and a combinatorial logic block, for example, which can be used to execute instructions by performing logical operations such as AND, OR, NOT, NAND, NOR, and XOR, and invert (e.g., NOT) logical operations on data (e.g., one or more operands). For example, functional unit circuitry may be used to perform arithmetic operations such as addition, subtraction, multiplication, and division on operands via a number of operations.

Artificial intelligence (AI) can be used in conjunction memory resources. AI can include a controller, computing device, or other system to perform a task that normally requires human intelligence. AI can include the use of one or more machine learning models. As described herein, the term “machine learning” refers to a process by which a computing device is able to improve its own performance through iterations by continuously incorporating new data into an existing statistical model. Machine learning can facilitate automatic learning for computing devices without human intervention or assistance and adjust actions accordingly.

### BRIEF DESCRIPTION OF THE DRAWINGS

FIG. 1 is a flow diagram representing an example method for treatment plan identification in accordance with a number of embodiments of the present disclosure.

FIG. 2 is another flow diagram representing an example method for treatment plan identification in accordance with a number of embodiments of the present disclosure.

FIG. 3 is a functional diagram representing a processing resource in communication with a memory resource having instructions written thereon in accordance with a number of embodiments of the present disclosure.

FIG. 4 is another functional diagram representing a processing resource in communication with a memory resource having instructions written thereon in accordance with a number of embodiments of the present disclosure.

FIG. 5 is yet another flow diagram representing an example method for treatment plan identification in accordance with a number of embodiments of the present disclosure.

FIG. 6 is another flow diagram representing an example method for treatment plan identification in accordance with a number of embodiments of the present disclosure.

FIG. 7 is yet another flow diagram representing an example method for treatment plan identification in accordance with a number of embodiments of the present disclosure.

### DETAILED DESCRIPTION

Apparatuses, machine-readable media, and methods related to treatment plan identification are described. Determining where and when to seek medical treatment can include determining where to go (e.g., hospital, clinic, virtual appointment, etc.), determining what treatment and/or services may be covered by insurance, and determining how to get to a provider (e.g., drive, ambulance, etc.), among others. When a decision is made, a patient may find that the provider they chose (e.g., an emergency room, a primary care physician, etc.) may have a long wait time or may not have medical devices available to treat the patient, for example. In such instances, the patient may leave the provider to seek treatment elsewhere, costing the patient time and money. As used herein, “health care provider” and “provider” may be used interchangeably.

Examples of the present disclosure can identify a health treatment plan for a patient by utilizing available data from the patient, providers, emergency vehicles, a cloud service, databases including generic health information, or a combination thereof. As used herein, a health treatment plan can include a diagnosis, a prescription, a transportation method, a treatment location, a provider, or any combination thereof for a patient. For instance, a health treatment plan may include a determination that based on information associated with a patient that the patient likely has an ear infection and can schedule a virtual (e.g., telemedicine) appointment. In another example, a health treatment plan may indicate a patient should travel by Ambulance A to Emergency Department A for treatment of a potential heart attack. A patient, for instance, can access an application on a mobile device and provide information about an ailment, and using information from a plurality of sources, a machine learning model can be utilized to determine a provider (e.g., a hospital, a specialist, etc.), and/or a transportation option, among others, that are a lowest cost (e.g., with or without insurance coverage), a shortest distance and/or shortest travel time, and/or have desired medical devices (e.g., X-ray, blood analysis, etc.), among others, to improve treatment results, customer satisfaction, or both.

Examples of the present disclosure can include a method for treatment plan identification including receiving at a first processing resource first signaling from a radio in communication with a second processing resource configured to monitor health data of a patient, receiving at the first processing resource second signaling from a radio in communication with a second processing resource configured to monitor data associated with a plurality of health care providers, and writing from the first processing resource to a memory resource coupled to the first processing resource data that is based at least in part on a combination of the first and the second signaling.

The method can include identifying at the first processing resource or a third, different processing resource output data representative of a health treatment plan for the patient based at least in part on input data representative of written information and generic health information stored in a portion of the memory resource or other storage accessible by the first processing resource and transmitting the output data representative of the health treatment plan via third signaling sent via a radio in communication with a fourth processing resource of a computing device accessible by the patient.

Other examples of the present disclosure can include a non-transitory machine-readable medium comprising a first processing resource in communication with a memory resource having instructions executable to receive at the first processing resource, the memory resource, or both, a plurality of input data from a plurality of sources, the plurality of sources comprising at least two of a mobile device of a patient, a medical device, a portion of the memory resource or other storage, an insurance network database, a health care provider network database, a volunteer health care provider network database, manually received input, an emergency vehicle network database, and environmental sensors and request additional input data from at least one of the plurality of sources. The medium can include instructions executable to write from the first processing resource to the memory resource the received input data and received additional input data, identify at the first processing resource or a second processing resource output data representative of a health treatment plan including a diagnosis for the patient, a prescription for the patient, a transportation method for the patient, a treatment location for the patient, an available volunteer provider, or any combination thereof based at least in part on input data representative of the data written from the first processing resource, and transmit the output data representative of the health treatment plan to the mobile device of the patient via signaling sent via a radio in communication with a third processing resource of the patient's mobile device.

Yet other examples of the present disclosure can include a same or different non-transitory machine-readable medium comprising a first processing resource in communication with a memory resource having instructions executable to receive at the first processing resource, the memory resource, or both, patient health data via first signaling configured to monitor patient health data, via signaling sent via a radio in communication with a processing resource of a mobile device of the patient, or both, receive at the first processing resource, the memory resource, or both, health care provider data via second signaling configured to monitor health care provider data including health care provider availability, health care provider cost, medical device availability, or a combination thereof, and receive at the first processing resource, the memory resource, or both, emergency vehicle data via third signaling configured to monitor emergency vehicle location and availability.

The medium can include instructions executable to write from the first processing resource to the memory resource the patient health data, heath care provider data, and emergency vehicle data, identify at the first processing resource or a second processing resource output data representative of a health treatment plan for the patient using a trained machine learning model, input data representative of the written patient health data, the written heath care provider data, and the written emergency vehicle data, and input data representative of a database of generic health data, and transmit, via a radio, the output data representative of the health treatment plan to the patient, a health care provider, an emergency vehicle, or any combination thereof.

In the following detailed description of the present disclosure, reference is made to the accompanying drawings that form a part hereof, and in which is shown by way of illustration how one or more embodiments of the disclosure can be practiced. These embodiments are described in sufficient detail to enable those of ordinary skill in the art to practice the embodiments of this disclosure, and it is to be understood that other embodiments can be utilized and that process, electrical, and structural changes can be made without departing from the scope of the present disclosure.

It is also to be understood that the terminology used herein is for the purpose of describing particular embodiments only and is not intended to be limiting. As used herein, the singular forms “a,” “an,” and “the” can include both singular and plural referents, unless the context clearly dictates otherwise. In addition, “a number of,” “at least one,” and “one or more” (e.g., a number of memory devices) can refer to one or more memory devices, whereas a “plurality of” is intended to refer to more than one of such things. Furthermore, the words “can” and “may” are used throughout this application in a permissive sense (i.e., having the potential to, being able to), not in a mandatory sense (i.e., must). The term “include,” and derivations thereof, means “including, but not limited to.” The terms “coupled,” and “coupling” mean to be directly or indirectly connected physically or for access to and movement (transmission) of commands and/or data, as appropriate to the context.

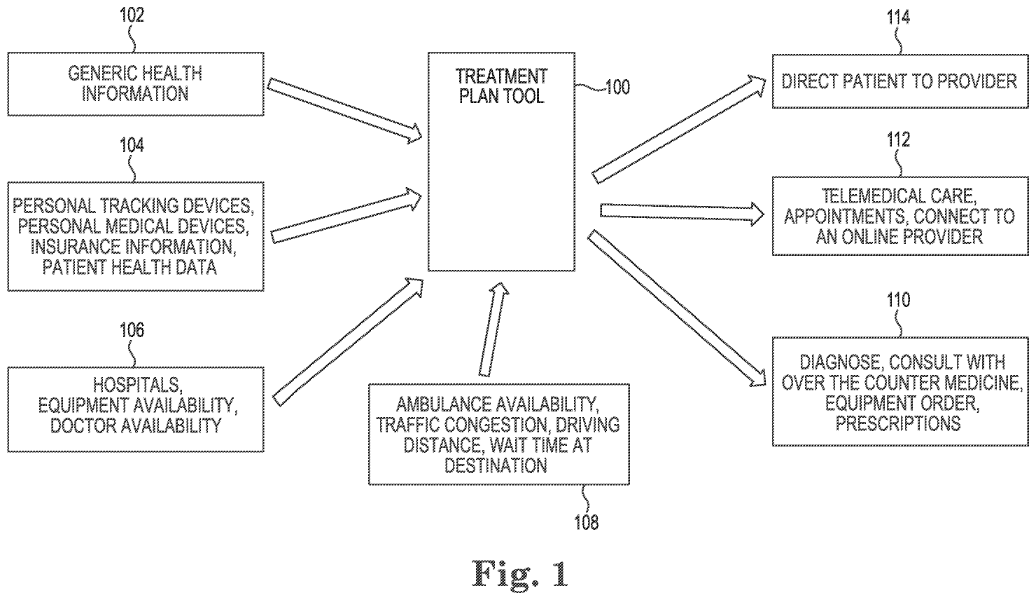

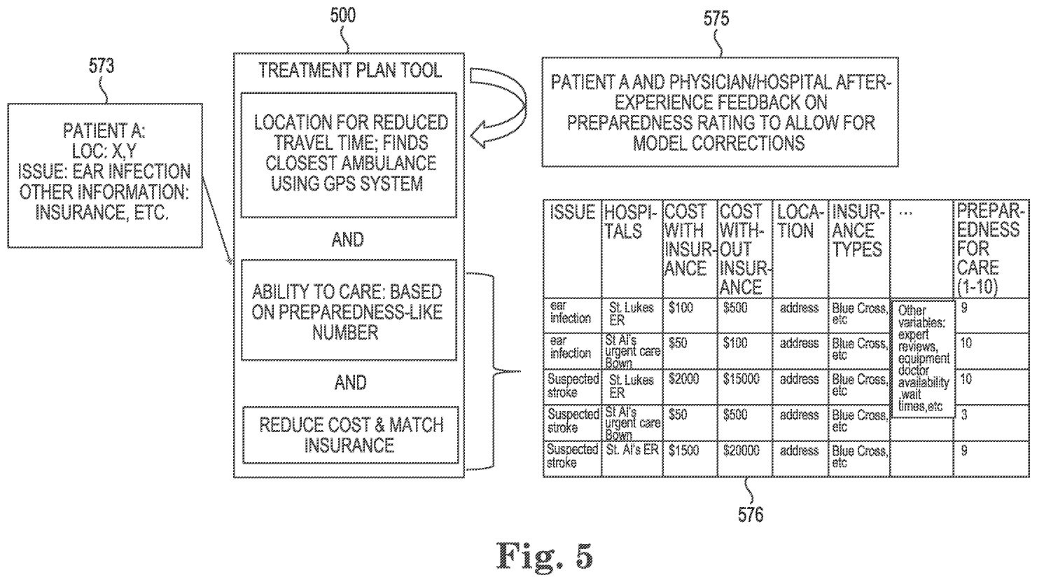

The figures herein follow a numbering convention in which the first digit or digits correspond to the figure number and the remaining digits identify an element or component in the figure. Similar elements or components between different figures can be identified by the use of similar digits. For example, 100 can reference element “00” in FIG. 1, and a similar element can be referenced as 500 in FIG. 5. As will be appreciated, elements shown in the various embodiments herein can be added, exchanged, and/or eliminated so as to provide a number of additional embodiments of the present disclosure. In addition, the proportion and/or the relative scale of the elements provided in the figures are intended to illustrate certain embodiments of the present disclosure and should not be taken in a limiting sense.

FIG. 1 is a flow diagram representing an example method for treatment plan identification in accordance with a number of embodiments of the present disclosure. The treatment plan tool 100 can be used to optimize a patient's health care experience by leveraging data from near devices (e.g., personal devices, medical devices, etc.). Optimization of a health care experience can include connecting patients with a best or most effective use of a situation or resource. For instance, optimization of the health care experience can include determining a fastest travel route, a least expensive treatment, a hospital with a shortest wait time, a provider specializing in a particular ailment, or a combination thereof. For instance, Hospital A may be farther away than Hospital B, but it may have a shorter emergency room wait time, making it the optimal choice.

The treatment plan tool 100 can include, in some examples, a processing resource in communication with a memory resource that utilizes AI to sort determine a health treatment plan. Put another way, the treatment plan tool 100 creates a plan of action for a patient based on data available to the treatment plan tool 100 including, but not limited to, patient health data, provider data, emergency vehicle data, insurance data, and a database of generic health data. The treatment plan tool 100 can facilitate communication between sources (e.g., devices, hospitals, etc.). For instance, the sources may not communicate directly with one another, but may share date with the treatment plan tool 100, which can in turn, communicate that shared data with other sources, where applicable.

The treatment plan tool 100 (and associated AI (e.g., including machine learning model(s)) can be trained using a training dataset. For instance, the training dataset can include a set of examples used to fit parameters of the AI. For instance, the training dataset can include data associated with patient health data, provider data, emergency vehicle data, insurance data, and generic health data. In some examples, the treatment plan tool 100 can also be trained using new input data (e.g., new data from patients, providers, insurance companies, emergency vehicle networks, research data, etc., among others).

As noted, the treatment plan tool 100 can receive input data from a plurality of sources. The input data can be encrypted, in some examples. Sources can include a database generic health information 102, patient health information sources 104 (e.g., personal tracking devices, personal medical devices, insurance information, patient health data, etc.), providers 106 (e.g., hospitals, equipment availability, doctor availability, etc.), and emergency vehicle networks and/or environmental sensors 108 (e.g., ambulance availability, traffic congestion, driving distance, wait time at destination, etc.), among others. For instance, the database of generic health information 102 may include common symptoms, visuals, treatments, and other data associated with common ailments such as conjunctivitis, ear infections, etc. Environmental sensors, for instance, can include weather sensors or cameras that may indicate road conditions (e.g., snow, ice), wind conditions (e.g., too windy for medical helicopter), or traffic conditions (e.g., roads closed), among others.

Patient data can be received from personal tracking devices (e.g., at 104) such as a global positioning service (GPS) on a mobile device including a patient location, a current travel speed, etc. Patient health data (e.g., at 104) can be received from personal medical devices (e.g., heartrate monitor, insulin pump, etc.) and/or insurance companies (e.g., current coverage, approved providers, etc.). In some examples, a patient can manually input patient data such as address or birthday information and/or patient health data such as current symptoms, current ailments, family health history, allergies, patient health history, etc. via an application on a computing device and associated with the treatment plan tool 100.

Provider information 106 received at the treatment plan tool 100 can include data associated with hospitals and other providers (clinics, urgent care locations, etc.). For instance, the data can include locations, bed availability, specialist availability, doctor or other provider availability, medical device/equipment availability, waiting room times, volunteer provider availability, etc. Emergency vehicle network data 108 received at the treatment plan tool 100 can include ambulance availability and locations, traffic reports and congestion, travel time including driving distance and or driving times, and wait times at the destination including either wait times inside the facility or wait times outside the facility (e.g., ambulance lines, etc.).

Using the data received from the plurality of sources, the treatment plan tool 100 can determine a treatment plan for the patient. Put another way, the treatment plan tool 100 can identify data representative of a health treatment plan for the patient using a machine learning model that considers the input data representative of patient health data, health insurance data, health care provider data, emergency vehicle data, generic health data, among others, or any combination thereof.

In some examples, the health treatment plan can include suggestions directing the patient to a particular provider 114. For instance, a closest hospital with a particular specialist that suits the patient's ailments may be suggested. In some instances, alternates to an in-person visit may be suggested at 112. For instance, a suggestion for telemedical care may be made, appointments for any type of care may be made, and/or a patient may be connected with an online provider for a virtual visit. In such an example, a patient may have conjunctivitis based on symptoms found in the generic health information database. The treatment plan tool 100 may be used to determine a virtual visit may be suitable with a provider, rather than an in-person visit, which may be more time-consuming or risky (e.g., during Covid-19 pandemic).

In some examples, the treatment plan tool 100 may output a diagnosis, suggest over-the-counter medication, order equipment, or suggest a prescription at 110. For instance, a patient's health data may indicate he or she is running low on diabetes testing supplies, the treatment plan tool 100 may order or suggest an order of the diabetes testing supplies. In another example, the patient may have symptoms of an ear infection, and a suggested prescription may be provided to the patient and/or the patients primary care physician.

In some examples, the treatment plan tool 100 and associated AI and memory resource or storage can be updated based on the data associated with the input as discussed herein. For instance, new patient symptoms, insurance data, patient health data, specialist information, etc. can be saved in the memory resource or storage and the treatment plan tool 100 can self-learn to update and improve accuracy and efficiency of treatment plan decisions.

In some examples, feedback can be requested regarding the treatment plan tool 100. The patient may be prompted to take a survey or leave feedback regarding ease of use, results, accuracy, visuals, usefulness, and performance of the treatment plan tool 100. Based on the received feedback, the treatment plan tool 100 can be adjusted. For instance, based on the feedback, the treatment plan tool 106 may be adjusted to improve a user interface, accessibility, visuals, etc.

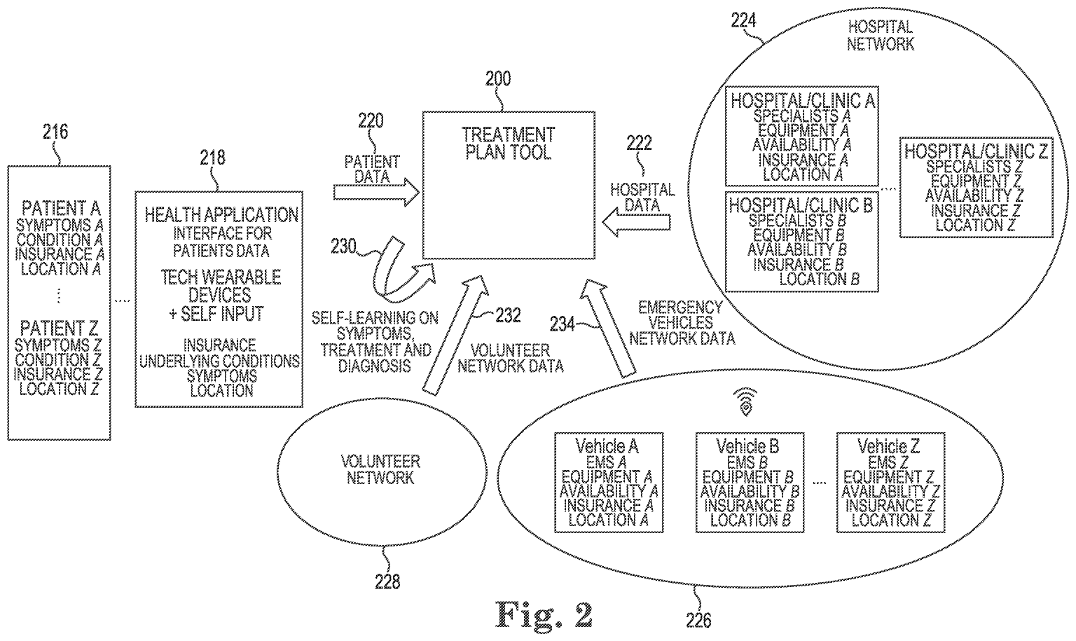

FIG. 2 is another flow diagram representing an example method for treatment plan identification in accordance with a number of embodiments of the present disclosure. FIG. 2 illustrates different sources and input data associated with the treatment plan tool 200. For instance, the treatment plan tool 200 can receive patient data 220, volunteer network data 232, emergency vehicle network data 234, and hospital/provider data 222. Patient data 220 can include data associated with the patient such as patient health data (e.g., treatment history, allergies, current ailments, medication lists, symptoms, etc.), current location data, address data, insurance information, birthdate, Social Security number, etc.

A number of patients 216 (e.g., Patient A, . . . , Patient Z) may each have their own patient data. For instance, Patient A may have symptoms A (e.g., chest pain, shortness of breath, etc.), conditions A (e.g., diabetes, high cholesterol, etc.), insurance A (e.g., coverage at all hospitals), and may be located at location A. Other patients, such as patient Z, may have some, all, or none of the same symptoms, conditions, insurances, and locations. The treatment plan tool 200 may receive patient data from the number of patients 216 via an application downloaded on a mobile device, such as a health application acting as an interface for patients' data. For instance, a patient's wearable device and/or monitors (e.g., smart watch, heart monitor, etc.) may provide data to the application or the patient may manually input data into the application (e.g., address, phone number, emergency contacts, underlying conditions, insurance information, current symptoms, current location, etc.). In some examples, a GPS or other location device on the mobile device may send location data to the application and/or treatment plan tool 200. The patient data 220 can be received at the treatment plan tool 200 and used in determining a health treatment plan.

In some examples, the treatment plan tool may receive volunteer network data 232 from a volunteer network 228. For instance, health care providers may volunteer to provide reduced-cost services to uninsured or underinsured patients. The treatment plan tool can receive information regarding specialists, costs, and availability, among other information. In such examples, the treatment plan tool 200 can receive the volunteer network data 232 and use that data in its determination of a heath treatment plan.

The treatment plan tool 200 can receive hospital/provider data 222. The hospital/provider data can be received from different health care providers 224 including, for instance, Hospital A, Hospital B, . . . , Hospital Z. The health care providers may include hospitals, clinics, urgent care facilities, or private practices, among others. In some examples, the health care providers are part of a network available to the treatment plan tool 200. The hospital/provider data 222 may include data for each health care provider including, for instance, specialists, equipment available (e.g., available X-ray machines, available ventilators, etc.), bed availability, insurance acceptance, and physical location. In such examples, the treatment plan tool 200 can receive the hospital/provider data 222 and use that data in its determination of a heath treatment plan.

In some examples, the treatment plan tool 200 can receive emergency vehicle network data 234. The emergency vehicle network data can be received from different emergency vehicles 226 including, for instance, Vehicle A, Vehicle B, . . . , Vehicle Z. The emergency vehicles may include ambulances, private transport, or other emergency vehicles. In some examples, the emergency vehicles are part of a network available to the treatment plan tool 200. The emergency vehicle network data 234 may include data for each emergency vehicle including, for instance, type of vehicle, available equipment on the vehicle, gurney availability, insurance acceptance, and physical location. In such examples, the treatment plan tool 200 can receive the emergency vehicle network data 234 and use that data in its determination of a heath treatment plan.

The treatment plan tool 200 can use the data received, along with previously received data to self-learn symptoms and associated treatments and diagnoses at 230, for instance, as part of a machine learning model. The treatment plan tool 200 can identify (e.g., at a processing resource) output data representative of a health treatment plan for the patient, which may include a diagnosis, a prescription, a transportation method, a treatment location, an available volunteer provider, or any combination thereof.

Returning to the example of Patient A within the number of patients 216, based on the received patient data 220, the treatment plan tool 200 may determine the patient should go to a hospital via an ambulance. In such an example, the treatment plan tool may suggest, for instance via the health application, that the patient can get to Hospital B via Vehicle Z in the fastest manner while being covered by insurance. It may also be determined that Hospital B has the shortest wait time and correct specialists, making it the optimal option for Patient A. Patient Z, who may have the same symptoms and conditions, may be sent elsewhere based on location, wait times, insurance acceptance, specialists, travel time, etc.

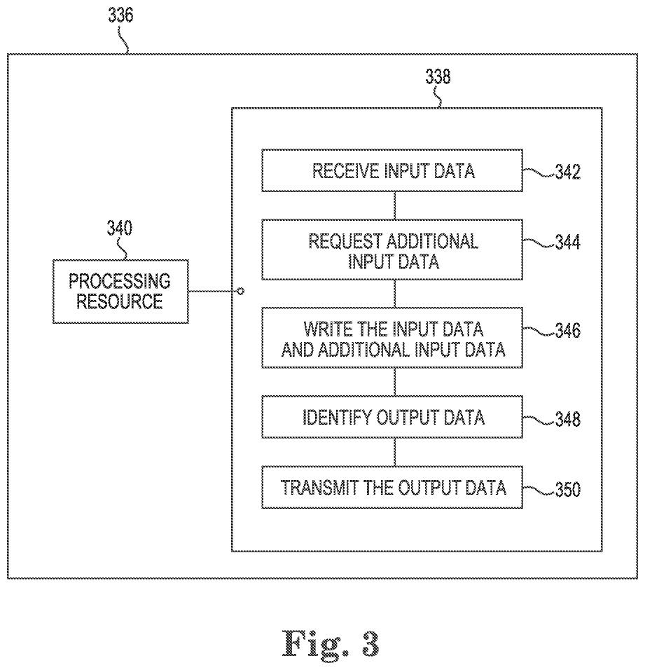

FIG. 3 is a functional diagram representing a processing resource 340 in communication with a memory resource 338 having instructions 342, 344, 346, 348, 350 written thereon in accordance with a number of embodiments of the present disclosure. In some examples, the processing resource 340 and memory resource 338 comprise a system 336 such as a treatment plan tool (e.g., treatment plan tool 100, 200, or 500 illustrated in FIGS. 1, 2, and 5, respectively).

The system 336 illustrated in FIG. 3 can be a server or a computing device (among others) and can include the processing resource 340. The system 336 can further include the memory resource 338 (e.g., a non-transitory MRM), on which may be stored instructions, such as instructions 342, 344, 346, 348, 350. Although the following descriptions refer to a processing resource and a memory resource, the descriptions may also apply to a system with multiple processing resources and multiple memory resources. In such examples, the instructions may be distributed (e.g., stored) across multiple memory resources and the instructions may be distributed (e.g., executed by) across multiple processing resources.

The memory resource 338 may be electronic, magnetic, optical, or other physical storage device that stores executable instructions. Thus, the memory resource 338 may be, for example, non-volatile or volatile memory. For example, non-volatile memory can provide persistent data by retaining written data when not powered, and non-volatile memory types can include NAND flash memory, NOR flash memory, read only memory (ROM), Electrically Erasable Programmable ROM (EEPROM), Erasable Programmable ROM (EPROM), and Storage Class Memory (SCM) that can include resistance variable memory, such as phase change random access memory (PCRAM), three-dimensional cross-point memory, resistive random access memory (RRAM), ferroelectric random access memory (FeRAM), magnetoresistive random access memory (MRAM), and programmable conductive memory, among other types of memory. Volatile memory can require power to maintain its data and can include random-access memory (RAM), dynamic random-access memory (DRAM), and static random-access memory (SRAM), among others.

In some examples, the memory resource 338 is a non-transitory MRM comprising Random Access Memory (RAM), an Electrically-Erasable Programmable ROM (EEPROM), a storage drive, an optical disc, and the like. The memory resource 338 may be disposed within a controller and/or computing device. In this example, the executable instructions 342, 344, 346, 348, 350 can be “installed” on the device. Additionally, and/or alternatively, the memory resource 338 can be a portable, external or remote storage medium, for example, that allows the system to download the instructions 342, 344, 346, 348, 350 from the portable/external/remote storage medium. In this situation, the executable instructions may be part of an “installation package”. As described herein, the memory resource 338 can be encoded with executable instructions for determining a health treatment plan.

The instructions 342, when executed by a processing resource such as the processing resource 340 (herein after referred to as the “first processing resource 340”), can include instructions to receive at the first processing resource 340, the memory resource 338, or both, a plurality of input data from a plurality of sources, the plurality of sources comprising at least two of a mobile device of a patient, a medical device, a portion of the memory resource or other storage, an insurance network database, a health care provider network database, a volunteer health care provider network database, manually received input, an emergency vehicle network database, and environmental sensors. In some examples, the plurality of input data can include patient health data, provider hospital bed availability, traffic data, emergency vehicle availability, specialist availability, insurance coverage data, treatment costs, volunteer health care provider availability, generic health data, medical device availability, travel times, or any combination thereof. For instance, data may be manually entered via an application of a mobile device for sending to the first processing resource 340 or automatically (e.g., with little or no human intervention) to the first processing resource 340.

The instructions 344, when executed by a processing resource such as the first processing resource 340, can include instructions to request additional input data from at least one of the plurality of sources. For instance, if a patient submits symptoms via the application, the first processing resource 340 may request additional information via the application in the form of follow-up questions to the patient (e.g., “Do you have a fever?”, “Do you have shortness of breath?”, etc.). In some examples, the instructions can be executable to identify, based on received input data from the mobile device of the patient, a threshold health data event, and responsive to the identification, request the additional input data, wherein the additional input data comprises data to supplement and compliment the received input data. For instance, the threshold health data event may include a blood pressure reading above a threshold level or a resting or active heartrate above a threshold level. In such an example, the patient may be prompted via the application with a request for additional input data and/or suggestions to seek treatment.

The instructions 346, when executed by a processing resource such as the processing resource 340, can include instructions to write from the first processing resource 340 to the memory resource 338 the received input data and received additional input data. Such data can be stored in the memory resource 338 for use in determining a health treatment plan for the patient.

The instructions 348, when executed by a processing resource such as the processing resource 340, can include instructions to identify at the first processing resource 340 or a second processing resource output data representative of a health treatment plan including a diagnosis for the patient, a prescription for the patient, a transportation method for the patient, a treatment location for the patient, an available volunteer provider, or any combination thereof based at least in part on input data representative of the data written from the first processing resource 340. In some examples, the instructions are executable to identify the output data representative of the health treatment plan based at least in part on generic health information stored in a portion of the memory resource 338 or other storage accessible by the first processing resource 340. For instance, a database of generic health information may be maintained and updated including common ailments, associated treatments, and associated symptoms.

In some instances, the identification includes the use of a trained machine learning model. For example, the trained machine learning model can use all or some of the input data to determine one or more health treatment plans for the patient. In some instances, the health treatment plans may be sorted based on optimization scores. For instance, a health treatment plan that is the least expensive and results in the quickest treatment may have a higher optimization score than a health treatment plan that does not include a desired specialist and is not covered by a patient's insurance.

The instructions 350, when executed by a processing resource such as the processing resource 340, can include instructions to transmit the output data representative of the health treatment plan to the mobile device of the patient via signaling sent via a radio in communication with a third processing resource of the patient's mobile device. For example, upon identification of the health treatment plan or health treatment plans, the patient may be alerted via the application. The alert may include instructions, in some examples, to execute the health treatment plan (e.g., chew an aspirin, wait for Emergency Vehicle A to arrive, Hospital B has been alerted, etc.).

In a non-limiting example, the instructions can be executable to identify at the first processing resource or the second processing resource output data representative of an additional health treatment plan including at least one different option for the diagnosis for the patient, the prescription for the patient, the transportation method for the patient, the treatment location for the patient, the available volunteer provider, or any combination thereof. Put another way, the patient may be provided with multiple treatment plans, as described above. The output data representative of the additional health treatment plan can be transmitted to the mobile device of the patient via signaling sent via the radio in communication with the third processing resource of the patient's mobile device, and the patient can be prompted, via a user interface of the patient's mobile device, to choose the output data representative of the health treatment plan or the output data representative of the additional health treatment plan. Responsive to the patient's choice, the output data representative of the health treatment plan or the output data representative of the additional health treatment plan can be displayed via the user interface.

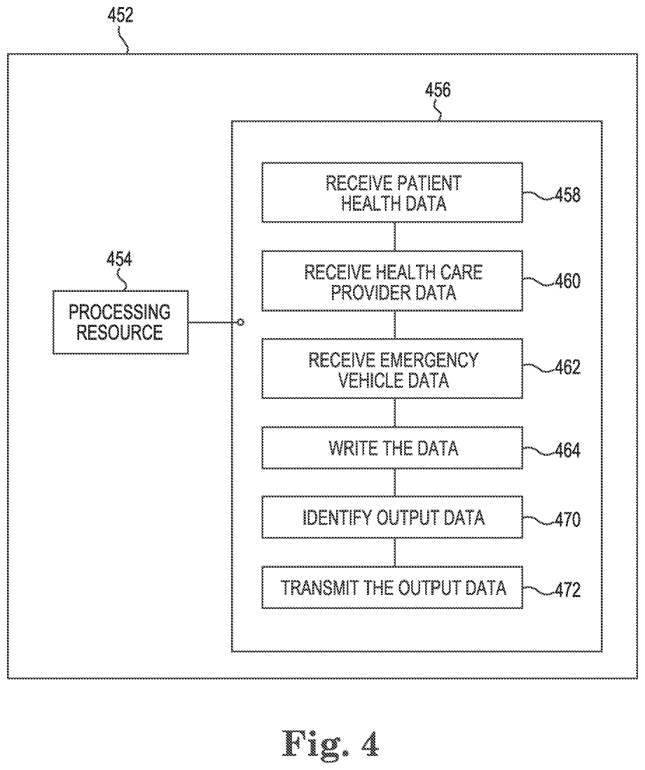

FIG. 4 is another functional diagram representing a processing resource 454 in communication with a memory resource 456 having instructions 458, 460, 462, 464, 470, 472 written thereon in accordance with a number of embodiments of the present disclosure. In some examples, the processing resource 454 (herein after referred to as “the first processing resource 454”) and the memory resource 456 may be analogous to processing resource 340 and memory resource 338, respectively, as described with respect to FIG. 3. In some examples, the processing resource 454 and the memory resource 456 comprise a system 452 such as treatment plan tool 100, 200, or 500 illustrated in FIGS. 1, 2, and 5, respectively.

The instructions 458, when executed by a processing resource such as the processing resource 454, can include instructions to receive at the first processing resource 454, the memory resource 456, or both, patient health data via first signaling configured to monitor patient health data, via signaling sent via a radio in communication with a processing resource of a mobile device of the patient, or both. For instance, the patient health data can be received from a heart monitor, insulin pump, smart watch, or other health monitoring device. The patient health data may be entered manually by a patient, for instance via an application on a mobile device. In some examples, the patient health data can include health symptoms, a health event (e.g., heart attack, high blood pressure, slow breathing, etc.), personal health information of the patient (e.g., preexisting conditions, allergies, etc.), identifying information of the patient (e.g., name, address, birthdate, etc.), a location of the patient, data collected by a health monitor (e.g., heart rate, etc.), health insurance data of the patient, manually input data of the patient, or any combination thereof.

The instructions 460, when executed by a processing resource such as the first processing resource 454, can include instructions to receive at the first processing resource 454, the memory resource 456, or both, health care provider data via second signaling configured to monitor health care provider data including health care provider availability, health care provider cost, medical device availability, or a combination thereof. For instance, hospitals and other health care providers can provide data that can be used to make health treatment plan decisions. Example data may include available X-ray machines, intensive care unit (ICU) bed availability, specialist availability, wait times, and procedure costs, among others.

The instructions 462, when executed by a processing resource such as the first processing resource 454, can include instructions to receive at the first processing resource 454, the memory resource 456, or both, emergency vehicle data via third signaling configured to monitor emergency vehicle location and availability. For instance, emergency vehicles can provide location data and equipment availability for used in determining a health treatment plan for a patient.

In some examples, the instructions are executable to receive at the first processing resource, the memory resource, or both, fourth signaling configured to monitor volunteer heath care provider availability. For example, health care providers willing to provide free or reduced-cost care may provide this data and their availability.

The instructions 464, when executed by a processing resource such as the first processing resource 454, can include instructions to write from the first processing resource 454 to the memory resource 456 the patient health data, heath care provider data, and emergency vehicle data. When included, volunteer health care provider availability may be written to the memory resource 456. In some examples, the memory resource 456 or storage can be updated using the written data. The updated memory resource 456 or storage, along with updates to AI can allow for self-learning and improved accuracy, efficiency, and consistency in health treatment plan determinations.

The instructions 470, when executed by a processing resource such as the first processing resource 454, can include instructions to identify at the first processing resource 454 or a second processing resource output data representative of a health treatment plan for the patient using a trained machine learning model, input data representative of the written patient health data, the written heath care provider data, and the written emergency vehicle data, and input data representative of a database of generic health data. The database, for instance, can be part of the memory resource 456 or other storage communicatively coupled to the medium and can include generic health symptoms and associated diagnoses and treatments.

In some examples, identifying the output data can include determining a diagnosis for the patient based on the patient health data and the database of generic health data, determining a prescription for the patient based on the patient health data and the database of generic health data, determining a transportation method for the patient based on the patient health data, the health care provider data, and the emergency vehicle data, determining a treatment location for the patient based on the patient health data, the health care provider data, and the emergency vehicle data, or any combination thereof. In some examples, identifying the output data can include scheduling an appointment with a health care provider. For instance, if a determination is made that the patient may not need immediate treatment, an appointment can be suggested or automatically made.

The instructions 472, when executed by a processing resource such as the processing resource 454, can include instructions to transmit, via a radio, the output data representative of the health treatment plan to the patient, a health care provider, an emergency vehicle, or any combination thereof. For instance, if it is determined the patient should be treated immediately, the treatment plan can be transmitted to the patient (e.g., via the application on the patient's mobile device), as well as to the emergency vehicle set to transport the patient and hospital set to receive the patient.

FIG. 5 is yet another flow diagram representing an example method for treatment plan identification in accordance with a number of embodiments of the present disclosure. FIG. 5 illustrates an example of a patient process using the treatment plan tool 500. At 573, Patient A provides patient data to the treatment plan tool 500. The patient data can include the patient's location (e.g., Location X, Y), the patient's symptoms or condition (e.g., ear infection), along with other information such as insurance coverage. At 500, the treatment plan 500, which may have access to a database of generic health data and also received data hospital/provider data, emergency vehicle data, and volunteer network data determines a quickest route to a health care provider, including finding an ambulance nearest Patient A based on location (e.g., using GPS). The treatment plan tool 500 can also determine a health care provider's ability to care for Patient A based on a preparedness-like number (e.g., an optimization score) and a cost to Patient A.

As illustrated in table 576, the treatment plan tool 500 may determine available health care providers (e.g., St. Luke's ER and St. Al's Urgent Care) and can provide costs, locations, accepted insurance plans, and other variables such as expert reviews, equipment availability, specialist availability, and wait times, among others. Using these factors, the treatment plan tool 500 can determined a preparedness number that ranks the health care providers for the patient. Patient A may choose from the options, or the application may automatically put a particular health treatment plan in motion based on the preparedness numbers.

At 575, the preparedness rating and performance of the treatment plan tool 500 can be evaluated. For instance, healthcare providers (e.g., paramedics in the ambulance, health care providers) and Patient A may give feedback as to the effectiveness, efficiency, and overall satisfaction with the health treatment plan and associated results. This information can be used to update a machine learning model associated with the treatment plan tool 500.

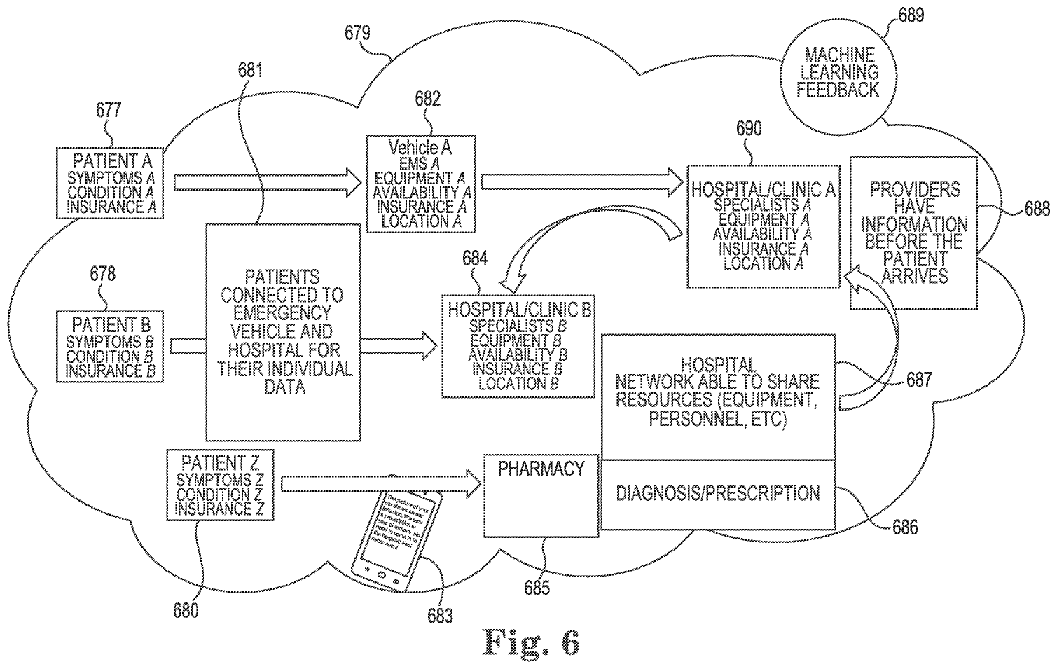

FIG. 6 is another flow diagram representing an example method for treatment plan identification in accordance with a number of embodiments of the present disclosure. FIG. 6 illustrates that data used to determine a health treatment plan can be shared via a cloud service 679, in some examples. Examples of the present disclosure can optimize a patient's health treatment plan to save time, money, and potentially lives. A treatment plan tool can use a machine learning model and its feedback 689 to make such determinations. For instance, as noted at 681, patients can be connected to emergency, vehicles, hospitals, or other health care providers that best suit their needs. This can reduce time spent searching for available providers, reduce time spent in waiting rooms, reduce costs incurred at multiple providers, reduce hospital and clinic resource expenses, reduce human error (e.g., utilizing generic health database), and improve patient outcomes, among others.

In an example, Patient A and his or her symptoms, conditions, and insurance 677 may indicate an emergency necessitating Vehicle A 382 (e.g., a medical helicopter) having particular equipment, services, locations, insurance acceptance, bed availability, etc. to transport Patient A to Hospital/Clinic A 690 having particular specialists, equipment, bed availability, insurance acceptance, and a particular location. In such an example, Patient A's health treatment plan can be transmitted to Patient A, Vehicle A, and Hospital A. The patient's health data such as current symptoms, name, birthdate, allergies, prior health conditions, etc.) can be available to health care providers in Vehicle A and at Hospital A before the patient arrives or is treated, which can increase treatment effectiveness and reduce errors.

Patient B and his or her symptoms, conditions, and insurance 678, may indicate a non-emergency trip to Hospital/Clinic B 684 may be suitable based on the available specialists, equipment, beds, insurance acceptance, location, etc. Patient C and his or her symptoms, conditions, and insurance 680 may indicate an ear infection with a prescription filled at a pharmacy 685 being suitable. That is, based on the input data and a database of generic health information, a diagnosis and/or prescription 686 can be determined. For instance, Patient Z may enter his or her symptoms into an application on his or her mobile device 683, and the health treatment plan may be displayed (e.g., “The picture of your ear shows an ear infection. We sent a prescription to your pharmacy. No need to come in to the hospital! Feel better soon!”).

As indicated by the arrows between Hospitals A, B, and C and at 687, information can be shared between health care providers, including resource information such as available equipment and personnel. For instance, Hospital A may be overwhelmed with patients needing ventilators, while Hospital B has several available. This information can be communicated between health care providers and patient loads or equipment can be shared. This information can be updated with the machine learning model to improve health treatment plan determinations.

In some examples, health care providers can be alerted of potential outbreaks based on collected data from a plurality of patients. For instance, if a plurality of patients within a threshold distance of each other indicate similar symptoms, are health care providers may be alerted of potential outbreaks. The health care providers can share data and resources to optimize health treatment plans in such examples.

FIG. 5 is yet another flow diagram representing an example method 590 for sharing data with a particular audience in accordance with a number of embodiments of the present disclosure. The method 590 can be performed by a system such as the systems described with respect to FIGS. 3 and 4. Similar to FIG. 1, data desired to be shared (e.g., first data) is referred to with respect to FIG. 5 as “particular input” so as to differentiate from other data referred to with respect to the description of FIG. 5.

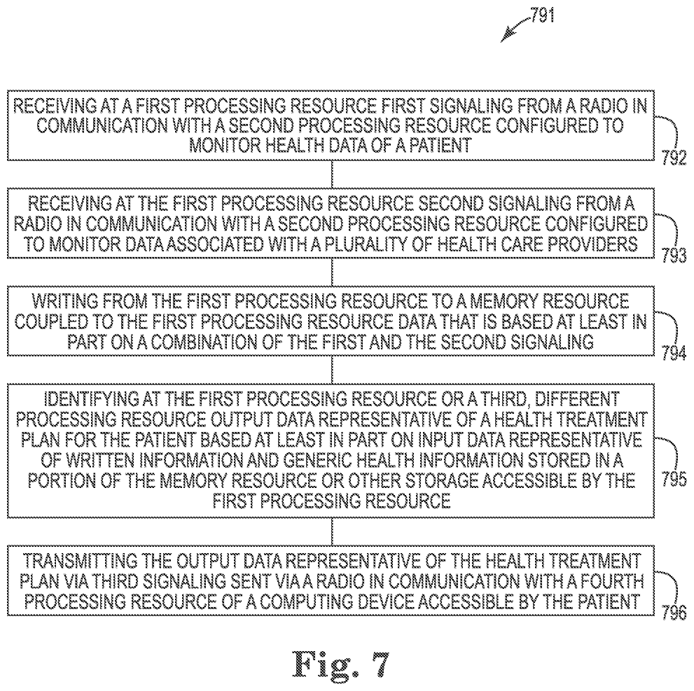

FIG. 7 is yet another flow diagram representing an example method 791 for treatment plan identification in accordance with a number of embodiments of the present disclosure. The method 791 can be performed by a system such as the systems described with respect to FIGS. 3 and 4.

At 792, the method 791 can include receiving at a first processing resource first signaling from a radio in communication with a second processing resource configured to monitor health data of a patient. The first signaling can include data associated with symptoms, ailments, height, weight, and/or other health data associated with the patient. At 793, the method 791 can include receiving at the first processing resource second signaling from a radio in communication with a second processing resource configured to monitor data associated with a plurality of health care providers. The second signaling can include data associated with provider availability, location, wait times, equipment availability, and/or other health care provider data.

The method 791, at 794, can include writing from the first processing resource to a memory resource coupled to the first processing resource data that is based at least in part on a combination of the first and the second signaling. The written data can be saved at the memory resource for use in determination of a current or future health treatment plan.

In some examples, the method 791 can include receiving at the first processing resource fourth signaling from a radio in communication with a fifth processing resource configured to monitor data associated with a plurality of emergency vehicles and writing from the first processing resource to a memory resource coupled to the first processing resource data that is based at least in part on a combination of the first, the second, and the fourth signaling. That is, emergency vehicle data can be factored into a health treatment plan determination. In some examples, a device may be located at an emergency vehicle and used for communication with the first processing resource. Data shared with a treatment plan tool by the device may be encrypted by the device.

In some examples, fifth signaling can be received from a radio in communication with a sixth processing resource configured to monitor data associated with a volunteer health care provider network, and data based at least in part on a combination of the first, the second, the fourth, and the fifth signaling can be written from the first processing resource to a memory resource coupled to the first processing resource. That is, volunteer network data can be factored into a health treatment plan determination.

At 795, the method 791 can include identifying at the first processing resource or a third, different processing resource output data representative of a health treatment plan for the patient based at least in part on input data representative of written information and generic health information stored in a portion of the memory resource or other storage accessible by the first processing resource. In some instances, identifying the output data can include utilizing a trained machine learning model to identify the output data representative of the health treatment plan based on data associated with the first and the second signaling, the generic health data, and previously received signaling and associated data associated with health treatment plans. For instance, data previous stored in the memory resource may be considered in determining a health treatment plan.

In some instances, identifying the output data includes determining a diagnosis for the patient, determining a prescription for the patient, determining a transportation method for the patient, determining a treatment location for the patient, or any combination thereof. In some examples, identifying the output data is based at least in part on feedback received at the first processing resource associated with outcomes of the output data representative of the health treatment plan. For example, following execution of a health treatment plan, the patient or involved health care providers may be asked to provide feedback on the overall experience. This feedback may be used in determining future health treatment plans.

At 796, the method 791 can include transmitting the output data representative of the health treatment plan via third signaling sent via a radio in communication with a fourth processing resource of a computing device accessible by the patient. For example, the patient can receive the health treatment plan and directions to execute the health treatment plan via an application on a mobile device. In some examples, the output data can be transmitted to at least one other receiver associated with a health care provider of the plurality of health care providers, an emergency vehicle, an insurance provider, a volunteer health care provider network, or any combination thereof. Put another way, those involved in the health treatment plan can be notified, so they can be prepared for treatment, including for instance, having access to patient health data before the patient arrives for treatment.

Although specific embodiments have been illustrated and described herein, those of ordinary skill in the art will appreciate that an arrangement calculated to achieve the same results can be substituted for the specific embodiments shown. This disclosure is intended to cover adaptations or variations of one or more embodiments of the present disclosure. It is to be understood that the above description has been made in an illustrative fashion, and not a restrictive one. Combination of the above embodiments, and other embodiments not specifically described herein will be apparent to those of skill in the art upon reviewing the above description. The scope of the one or more embodiments of the present disclosure includes other applications in which the above structures and processes are used. Therefore, the scope of one or more embodiments of the present disclosure should be determined with reference to the appended claims, along with the full range of equivalents to which such claims are entitled.

In the foregoing Detailed Description, some features are grouped together in a single embodiment for the purpose of streamlining the disclosure. This method of disclosure is not to be interpreted as reflecting an intention that the disclosed embodiments of the present disclosure have to use more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

What is claimed is:

1. A method, comprising:
receiving at a first processing resource first signaling from a radio in communication with a second processing resource configured to monitor health data of a patient;
receiving at the first processing resource second signaling from a radio in communication with a second processing resource configured to monitor data associated with a plurality of health care providers;
writing from the first processing resource to a memory resource coupled to the first processing resource data that is based at least in part on a combination of the first and the second signaling;
identifying at the first processing resource or a third, different processing resource output data representative of a health treatment plan for the patient based at least in part on input data representative of written information and generic health information stored in a portion of the memory resource or other storage accessible by the first processing resource; and
transmitting the output data representative of the health treatment plan via third signaling sent via a radio in communication with a fourth processing resource of a computing device accessible by the patient.

2. The method of claim 1, further comprising transmitting, via the third signaling, the output data representative of the health treatment plan to at least one other receiver associated with a health care provider of the plurality of health care providers, an emergency vehicle, an insurance provider, a volunteer health care provider network, or any combination thereof.

3. The method of claim 1 further comprising:
receiving at the first processing resource fourth signaling from a radio in communication with a fifth processing resource configured to monitor data associated with a plurality of emergency vehicles; and
writing from the first processing resource to a memory resource coupled to the first processing resource data that is based at least in part on a combination of the first, the second, and the fourth signaling.

4. The method of claim 2, further comprising:
receiving at the first processing resource fifth signaling from a radio in communication with a sixth processing resource configured to monitor data associated with a volunteer healthcare provider network; and
writing from the first processing resource to a memory resource coupled to the first processing resource data that is based at least in part on a combination of the first, the second, the fourth, and the fifth signaling.

5. The method of claim 1, wherein identifying the output data representative of the health treatment plan comprises utilizing a trained machine learning model to identify the output data representative of the health treatment plan based on data associated with the first and the second signaling, the generic health data, and previously received signaling and associated data associated with health treatment plans.

6. The method of claim 1, further comprising identifying the output data representative of the health treatment plan based at least in part on feedback received at the first processing resource associated with outcomes of the output data representative of the health treatment plan.

7. The method of claim 1, wherein identifying the output data representative of the health treatment plan comprises determining a diagnosis for the patient, determining a prescription for the patient, determining a transportation method for the patient, determining a treatment location for the patient, or any combination thereof.

8. The method of claim 1 further comprising:
receiving at the first processing resource via an application of a mobile device of the patient, manual input from the patient comprising personal patient data, patient health data, or a combination thereof; and
writing from the first processing resource to a memory resource coupled to the first processing resource data that is based at least in part on a combination of the first signaling, the second signaling, and the manual input.

9. A non-transitory machine-readable medium comprising a first processing resource in communication with a memory resource having instructions executable to:
receive at the first processing resource, the memory resource, or both, a plurality of input data from a plurality of sources, the plurality of sources comprising at least two of a mobile device of a patient, a medical device, a portion of the memory resource or other storage, an insurance network database, a health care provider network database, a volunteer health care provider network database, manually received input, an emergency vehicle network database, and environmental sensors;
request additional input data from at least one of the plurality of sources;
write from the first processing resource to the memory resource the received input data and received additional input data;
identify at the first processing resource or a second processing resource output data representative of a health treatment plan including a diagnosis for the patient, a prescription for the patient, a transportation method for the patient, a treatment location for the patient, an available volunteer provider, or any combination thereof based at least in part on input data representative of the data written from the first processing resource; and
transmit the output data representative of the health treatment plan to the mobile device of the patient via signaling sent via a radio in communication with a third processing resource of the patient's mobile device.

10. The medium of claim 9, wherein the instructions executable to request additional input data comprise the instructions executable to:
identify, based on received input data from the mobile device of the patient, a threshold health data event; and
responsive to the identification, request the additional input data, wherein the additional input data comprises data to supplement and compliment the received input data.

11. The medium of claim 9, further comprising the instructions executable to identify the output data representative of the health treatment plan based at least in part on generic health information stored in a portion of the memory resource or other storage accessible by the first processing resource.

12. The medium of claim 9, wherein the plurality of input data comprises patient health data, provider hospital bed availability, traffic data, emergency vehicle availability, specialist availability, insurance coverage data, treatment costs, volunteer health care provider availability, generic health data, medical device availability, travel times, or any combination thereof.

13. The medium of claim 9, further comprising the instructions executable to identify at the first processing resource or the second processing resource output data representative of the health treatment plan using a trained machine learning model.

14. The medium of claim 9, further comprising the instructions executable to:
identify at the first processing resource or the second processing resource output data representative of an additional health treatment plan including at least one different option for the diagnosis for the patient, the prescription for the patient, the transportation method for the patient, the treatment location for the patient, the available volunteer provider, or any combination thereof;
transmit the output data representative of the additional health treatment plan to the mobile device of the patient via signaling sent via the radio in communication with the third processing resource of the patient's mobile device;
prompt the patient, via a user interface of the patient's mobile device, to choose the output data representative of the health treatment plan or the output data representative of the additional health treatment plan; and
responsive to the patient's choice, display the output data representative of the health treatment plan or the output data representative of the additional health treatment plan via the user interface.

15. A non-transitory machine-readable medium comprising a first processing resource in communication with a memory resource having instructions executable to:
receive at the first processing resource, the memory resource, or both, patient health data via first signaling configured to monitor patient health data, via signaling sent via a radio in communication with a processing resource of a mobile device of the patient, or both;
receive at the first processing resource, the memory resource, or both, health care provider data via second signaling configured to monitor health care provider data including health care provider availability, health care provider cost, medical device availability, or a combination thereof;
receive at the first processing resource, the memory resource, or both, emergency vehicle data via third signaling configured to monitor emergency vehicle location and availability;
write from the first processing resource to the memory resource the patient health data, heath care provider data, and emergency vehicle data;
identify at the first processing resource or a second processing resource output data representative of a health treatment plan for the patient using a trained machine learning model, input data representative of the written patient health data, the written heath care provider data, and the written emergency vehicle data, and input data representative of a database of generic health data; and
transmit, via a radio, the output data representative of the health treatment plan to the patient, a health care provider, an emergency vehicle, or any combination thereof.

16. The medium of claim 15, further comprising the instructions executable to receive at the first processing resource, the memory resource, or both, fourth signaling configured to monitor volunteer heath care provider availability.

17. The medium of claim 15, wherein the patient health data comprises health symptoms, a health event, personal health information of the patient, identifying information of the patient, a location of the patient, data collected by a health monitor, health insurance data of the patient, manually input data of the patient, or any combination thereof.

18. The medium of claim 15, wherein the instructions executable to identify the output data representative of the health treatment plan comprise instructions executable to:
determine a diagnosis for the patient based on the patient health data and the database of generic health data;
determine a prescription for the patient based on the patient health data and the database of generic health data;
determine a transportation method for the patient based on the patient health data, the health care provider data, and the emergency vehicle data;
determine a treatment location for the patient based on the patient health data, the health care provider data, and the emergency vehicle data; or
any combination thereof.

19. The medium of claim 15, wherein the instructions executable to identify the output data representative of the health treatment plan comprise instructions executable to schedule an appointment with a health care provider.

20. The medium of claim 15, wherein the database of generic health data is part of the memory resource or other storage communicatively coupled to the medium and comprises generic health symptoms and associated diagnoses and treatments.
